(19) United States
(12) Patent Application Publication
(10) Pub. No.: US 2007/0104787 A1
(43) Pub. Date: May 10, 2007

(54) Carboxyalkyl cellulose esters for sustained delivery of pharmaceutically active substances

(76) Inventors: Jessica Dee Posey-Dowty, Kingsport, TN (US); Thelma Lee Watterson, Richmond, VA (US); Kevin Joseph Edgar, Kingsport, TN (US); Shane Kipley Kirk, Church Hill, TN (US); Michael Lee Welty, Kingsport, TN (US); Jinghua Yuan, Kingsport, TN (US); Michael Charles Shelton, Kingsport, TN (US); Larry Ronnie Lingerfelt JR., Blountville, TN (US); Alan Kent Wilson, Kingsport, TN (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/592,129

(22) Filed: Nov. 3, 2006

Related U.S. Application Data
(60) Provisional application No. 60/733,382, filed on Nov. 4, 2005.

(51) Int. Cl. A61K 31/717; A61K 9/22

(52) U.S. Cl. 424/468; 514/57

(57) ABSTRACT

Disclosed herein are pharmaceutical compositions comprising carboxyalkylcellulose esters for sustained delivery of pharmaceutically active substances. The composition comprises: at least one pharmaceutically active agent, and at least one carboxyalkyl cellulose ester comprising an anhydroglucose repeat unit having the structure: 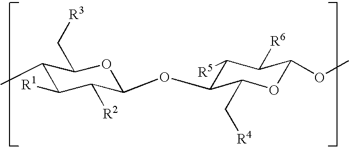
wherein: R¹-R⁶ are each independently selected from —OH, —OC(O)(alkyl), and —O(CH₂)ₓC(O)OH, and pharmaceutically acceptable salts thereof, wherein x ranges from 1-3, a degree of substitution per anhydroglucose of —OH ranges from 0.1 to 0.7, a degree of substitution per anhydroglucose of —C(O)(alkyl) ranges from 0.1 to 2.7, and a degree of substitution per anhydroglucose of —O(CH₂)ₓC(O)OH ranges from 0.2 to 0.75, and wherein in pharmaceutically acceptable media, the composition exhibits sustained release of the at least one pharmaceutically active agent. Also disclosed are methods of administering the compositions for sustained delivery, such as delivery following zero order kinetics.

This application claims the benefit of priority of U.S. Provisional Patent Application No. 60/733,382 filed Nov. 4, 2005.

### FIELD OF THE INVENTION

Disclosed herein are pharmaceutical compositions comprising carboxyalkyl cellulose esters for sustained delivery of pharmaceutically active substances. Also disclosed are methods of administering the compositions for sustained delivery, such as delivery following zero order kinetics.

### BACKGROUND

The pharmaceutical industry has an interest in controlled release of pharmaceutical agents. A number of controlled release dosage forms are-known, including matrix tablet systems incorporating active ingredients, fillers and various types of excipients. The wide range of properties of pharmaceutically active ingredients has given rise to the development of a number of different drug delivery systems using polymer technology to provide release of a particular medicament administration to a patient, such as after oral ingestion by a patient.

However, there remains a need to develop improved compositions providing sustained delivery of pharmaceutically active agents, such as compositions capable of slowing down or stopping the release of water soluble pharmaceutical actives at gastric pH while allowing sustained release over a suitable time at intestinal pH.

### BRIEF DESCRIPTION OF THE DRAWINGS

FIG. 1 is a plot of aspirin released (y-axis) versus time (minutes, x-axis) for Comparative Formulations C1-C3 in Example 1;

FIG. 2 is a plot of aspirin released (y-axis) versus time (minutes, x-axis) for Comparative Formulation C4 in Example 1;

FIG. 3 is a plot of aspirin released (y-axis) versus time (minutes, x-axis) for Comparative Formulations C5-C7 in Example 1;

FIG. 4 is a plot of aspirin released (y-axis) versus time (minutes, x-axis) for Formulations E1-E3 in Example 1;

FIG. 5 is a plot of aspirin released (y-axis) versus time (minutes, x-axis) for Formulation E4 in Example 1;

FIG. 6 is a plot of aspirin released (y-axis) versus time (minutes, x-axis) for Formulation E5 in Example 1;

FIG. 7 is a plot of aspirin released (y-axis) versus time (minutes, x-axis) for Formulation E6 in Example 1;

FIG. 8 is a plot of aspirin released (y-axis) versus time (minutes, x-axis) for Formulation E7 in Example 1;

FIG. 9 is a plot of aspirin released (y-axis) versus time (minutes, x-axis) for Formulation E8 in Example 1;

FIG. 10 is a plot of aspirin released (y-axis) versus time (minutes, x-axis) for Formulation E9 in Example 1;

FIG. 11 is a plot of aspirin released (y-axis) versus time (minutes, x-axis) for Formulation E10 in Example 1;

FIG. 12 is a plot of aspirin released (y-axis) versus time (minutes, x-axis) for Formulation E11 in Example 1;

FIG. 13 is a plot of aspirin released (y-axis) versus time (minutes, x-axis) for Formulation E12 in Example 1;

FIG. 14 is a plot of aspirin released (y-axis) versus time (minutes, x-axis) for Formulation E13 in Example 1;

FIG. 15 is a plot of aspirin released (y-axis) versus time (minutes, x-axis) for Formulation E14 in Example 1;

FIG. 16 is a plot of aspirin released (y-axis) versus time (minutes, x-axis) for Formulation E15 in Example 1;

FIG. 17 is a plot of aspirin released (y-axis) versus time (minutes, x-axis) for Formulation E16 in Example 1;

FIG. 18 is a ternary plot showing statistical results of % aspirin release at aspirin at pH 1.2 for 3 hours, for the Formulations E1-16;

FIG. 19 is a ternary plot showing statistical results of % aspirin release at pH 6.8 for 24 hours, for the Formulations E1-16;

FIG. 20 is a plot of trimethoprim, sulfamethizole, and levofloxacin released (y-axis) versus time (minutes, x-axis) for Formulations E17-19, respectively, in Example 3;

FIG. 21 is a plot of ibuprofen released (y-axis) versus time (minutes, x-axis) for Formulation E20 in Example 3;

FIG. 22 is a plot of ibuprofen released (y-axis) versus time (minutes, x-axis) for Comparative Formulation C8 in Example 1;

FIG. 23 is a plot of amiloride, fluconazole, and fexofenadine released (y-axis) versus time (minutes, x-axis) for Formulations E21-23, respectively, in Example 3; and

FIG. 24 is a plot of fexofenadine released from CMCAB solid dispersion (y-axis) versus time (minutes, x-axis) for Formulation E24 in Example 4.

### DETAILED DESCRIPTION

The present disclosure provides compositions comprising carboxyalkyl cellulose esters for sustained delivery of a pharmaceutically active agent. In one embodiment, carboxyalkyl cellulose esters combined with water soluble pharmaceutical actives may improve the sustained release of water soluble actives at intestinal pH while substantially preventing release at gastric pH. The combination could either be a matrix formulation as in a compression tablet or combined at a molecular level in a solid dispersion to provide the desired release profiles.

One embodiment disclosed herein provides a sustained release pharmaceutical composition comprising:
at least one pharmaceutically active agent, and
at least one carboxyalkylcellulose ester comprising an anhydroglucose repeat unit having the structure: 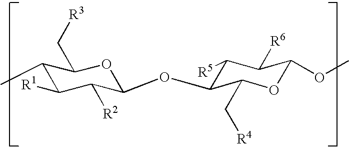
wherein:
R¹-R⁶ are each independently selected from —OH, —OC(O)(alkyl), and —O(CH₂)ₓC(O)OH, and pharmaceutically acceptable salts thereof, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75, and
wherein in pharmaceutically acceptable media, the composition exhibits sustained release of the at least one pharmaceutically active agent.

“Degree of substitution” as used herein refers to a number of substituents per anhydroglucose. A theoretical maximum degree of substitution is 3 is assumed unless stated otherwise as in HS-CMC (high solids carboxymethylcellulose) esters or low molecular weight CMC esters, which can have a maximum degree of substitution per anhydroglucose unit of greater than 3.0.

In one embodiment, the pharmaceutically acceptable salts include pharmaceutically acceptable salts of —OH and —O(CH₂)ₓC(O)OH having the structure O⁻A⁺ and —O(CH₂)ₓC(O)O⁻A⁺, respectively, wherein A⁺ is a counterion. Exemplary counterions include monovalent inorganic cations, such as lithium, sodium, potassium, rubidium, cesium, silver, divalent inorganic cations, such as magnesium, calcium, nickel, zinc, iron copper, or manganese, and ammonium and alkylammonium counterions. The counterion A⁺ need not necessarily be the same throughout the molecule and comprise a combination of differing counterions, as readily understood by one of ordinary skill in the art.

In one embodiment, “sustained release” refers to a sustained delivery (i.e., substantially continuous release) of the pharmaceutically active agent over time, such as a time of at least 4 h, e.g., a time ranging from 4-24 h, from 12-24 h, from 6-12 h, or even greater than 24 h, e.g., 1-5 days.

In one embodiment, the sustained release follows zero order kinetics, i.e., “zero order release.” In one embodiment, “zero order release” is indicated by a substantially linear plot of released pharmaceutically active agent over time, where “substantially linear” refers to a correlation coefficient (R) of at least 0.8, for a given time, such as a correlation coefficient of at least 0.9, or at least 0.95.

In one embodiment, the —OC(O)(alkyl) is chosen from —OC(O)(C₁-C₂₁, alkyl), such as —OC(O)(C₁-C₁₁ alkyl), —OC(O)(C₁-C₅ alkyl), or —OC(O)(C₁-C₃ alkyl). Alternatively, the —OC(O)(C₁-C₂₁ alkyl) can be referred to as a C₂-C₂₂ ester of a carboxyalkylcellulose ester.

In one embodiment, the carboxyalkylcellulose ester is chosen from carboxymethylcellulose esters. Exemplary carboxyalkylcellulose esters, include, but are not limited to carboxymethylcellulose acetate butyrate (CMCAB) (such as CMCAB-641-0.5 from Eastman Chemical Company), high solids CMCAB (HS-CMCAB), carboxymethylcellulose butyrate (CMCB), carboxymethylcellulose acetate propionate (CMCAP), high solids CMCAP (HS-CMCAP), carboxymethylcellulose propionate (CMCP), carboxymethylcellulose acetate (CMCA), carboxymethylcellulose acetate isobutryate (CMCAiB), carboxymethylcellulose isobutryate (CMCiB), carboxymethylcellulose acetate butyrate succinate, carboxymethylcellulose acetate butyrate maleate, carboxymethylcellulose acetate butyrate trimellitate.

In one embodiment, the at least one carboxyalkylcellulose ester is carboxymethylcellulose propionate having a degree of substitution per anhydroglucose of —OC(O)CH₂CH₃ ranging from 1.5 to 2.7.

In another embodiment, the at least one carboxyalkylcellulose ester is carboxymethylcellulose butyrate having a degree of substitution per anhydroglucose of —OC(O)CH₂CH₂CH₃ ranging from 1.5 to 2.7.

In yet another embodiment, the at least one carboxyalkylcellulose ester is carboxymethylcellulose acetate propionate having a degree of substitution per anhydroglucose of —OC(O)CH₃ ranging from 0.1 to 2.65 and a degree of substitution per anhydroglucose of —OC(O)CH₂CH₂H₃ ranging from 0.1 to 2.6.

In another embodiment, the at least one carboxyalkylcellulose ester is carboxymethylcellulose acetate butyrate having a degree of substitution per anhydroglucose of —OC(O)CH₃ ranging from 0.1 to 1.65 and a degree of substitution per anhydroglucose of —OC(O)CH₂CH₂H₃ ranging from 0.1 to 2.6.

In one embodiment, the pharmaceutically acceptable medium is chosen from water, acidic aqueous buffers, neutral aqueous buffers, basic aqueous buffers, and natural and simulated bodily fluids, such as gastric fluid (with or without pepsin), or intestinal fluid (with or without pancreatin).

In one embodiment, in pharmaceutically acceptable media, the composition exhibits release of the pharmaceutically active agent at a target pH. In one embodiment, the target pH is at least 5, such as a pH of at least 6, or a pH of at least 6.5. In one embodiment, release of the pharmaceutically active agent is stopped or released at a very slow rate at gastric pH (e.g., approximately 1.2), whereas sustained release as described herein occurs at intestinal pH (e.g., approximately 6.8) over a suitable time.

Accordingly, another embodiment disclosed herein provides a method of delivering at least one pharmaceutically active agent to a mammal, comprising:
(a) administering to the mammal a therapeutically effective amount of at least one pharmaceutically active agent with at least one carboxyalkylcellulose ester comprising an anhydroglucose repeat unit having the structure: 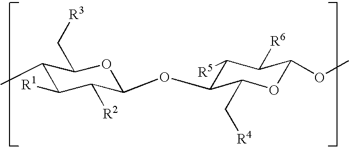
wherein:
R¹-R⁶ are each independently selected from —OH, —OC(O)(alkyl), and —O(CH₂)ₓC(O)OH, and pharmaceutically acceptable salts thereof, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75;
(b) releasing the at least one pharmaceutically active agent at gastric pH; and
(c) allowing sustained release of the at least one pharmaceutically active agent at intestinal pH.

In one embodiment, the pharmaceutically active agent is chosen from any suitable drug known in the art, such as those chosen from the classes of drugs including, for example, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthihes.

In one embodiment, the pharmaceutically active agent is chosen from those intended for oral administration. A description of these classes of drugs and a listing of species within each class can be found in Martindale, the extra Pharmacopoeia, Twenty-ninth Edition, the Pharmaceutical Press, London, 1989, the disclosure of which is incorporated herein by reference. The drug substances are commercially available and/or can be prepared by techniques known in the art.

Exemplary nutraceuticals and dietary supplements can also be included, such as those disclosed in, for example, Roberts et al., Nutraceuticals: The Complete Encyclopedia of Supplements, Herbs, Vitamins, and Healing Foods (American Nutraceutical Association, 2001), which is specifically incorporated by reference. A nutraceutical or dietary supplement, also known as phytochemicals or functional foods, is generally any one of a class of dietary supplements, vitamins, minerals, herbs, or healing foods that have medical or pharmaceutical effects on the body. Exemplary nutraceuticals or dietary supplements include, but are not limited to, folic acid, fatty acids (e.g., DHA and ARA), fruit and vegetable extracts, vitamin and mineral supplements, phosphatidylserine, lipoic acid, melatonin, glucosamine/chondroitin, Aloe Vera, Guggul, glutamine, amino acids (e.g., iso-leucine, leucine, lysine, methionine, phenylanine, threonine, tryptophan, and valine), green tea, lycopene, whole foods, food additives, herbs, phytonutrients, antioxidants, flavonoid constituents of fruits, evening primrose oil, flax seeds, fish and marine animal oils, and probiotics. Nutraceuticals and dietary supplements also include bio-engineered foods genetically engineered to have a desired property, also known as pharmafoods.

In one embodiment, the pharmaceutically active agent is soluble in pharmaceutically acceptable media. A suitable solubility for pharmaceutical applications can be readily determined by one of ordinary skill in the art. In one embodiment, a “soluble” drug is determined by the Biopharmaceutics Classification System (BCS). (Amidon, G. L.; Lennernas, H.; Shah, V. P.; Crison, J. R. “A Theoretical Basis for a Biopharmaceutic Drug Classification. The Correlation of in Vitro Drug Product Dissolution and in Vivo Bioavailability, Pharm. Res. 1995, 12(3), 413-420; Lennernas, H.; Abrahamsson, B. “he Use of Biopharmaceutic Classification of Drugs in Drug Discovery and Development: Current Status and Future Extension,” J Pharmacy and Pharmacology, 2005, 57(3), 273-285; u, C.-Y.; Benet, L. Z., “Predicting Drug Disposition via Application of BCS: Transport/Absorption/Elimination Interplay and Development of a Biopharmaceutics Drug Disposition Classification System,” Pharm. Res. 2005, 22(1),11-23;Dressman, J.; Butler, J.; Hempenstall, J.; Reppas, C. “The BCS: Where do we go from here?” Pharmaceutical Technology North America 2001, 25(7), 68-76.)

Exemplary pharmaceutically active agents include, but are not limited to, those chosen from aspirin, ibuprofen, fexofenadine, trimethoprim, sulfamethizole, amiloride, fluconazole, and fexofenadine, and salts thereof.

In one embodiment, the composition comprises:
(a) at least one carboxyalkyl cellulose in an amount ranging from 0.1 to 99 weight percent, based on the total weight (a) and (b) in said composition;
(b) the at least one pharmaceutically active agent in an amount ranging from 0.1 to 99 weight percent, based on the total weight (a) and (b) in said composition; and
(c) at least one additive chosen from plasticizers and flow aids in an amount ranging from 0 to 50 weight percent, based on the total weight of (a), (b), and (c) in the composition;
(d) an organic solvent, aqueous solvent, including but not limited to water, or a solvent mixture;
wherein the total weight of (a) and (b) is about 5 to 95 weight percent of the total weight of (a), (b), (c), and (d).

Exemplary plasticizers include, but are not limited to, Vitamin E TPGS, triethyl citrate, polyethylene glycol, diethyl phthalate, dibutyl sebacate, triacetin, sorbitol, propylene glycol, benzyl phenyl formate, chlorobutanol, glucose acetate, glucose acetate butyrate, glucose butyrate, glucose propionate, glucose acetate propionate, and glucose propionate butyrate.

In one embodiment, the pharmaceutical composition can include at least one other pharmaceutically acceptable additive, including one or more binding agents, filling agents, lubricating agents, suspending agents, sweeteners, flavoring agents, preservatives, buffers, wetting agents, disintegrants, effervescent agents, diluent and other excipients. Such excipients are known in the art.

Examples of filling agents are lactose monohydrate, lactose anhydrous, mannitol, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (SMCC).

Suitable lubricants, including agents that act on the flowability of the powder to be compressed, are colloidal silicon dioxide, such as Aerosil® 200; talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; maltodextrin; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

The pharmaceutical composition can take a variety of forms, including, for example, those chosen from tablets, hard and soft gelatin capsules, lozenges and troches, sachets, powders, and sprinkles. The composition can be formulated into a-dosage form for oral, rectal, intravaginal, injectable, pulmonary, nasal, buccal, topical, local, intracisternal, intraperitoneal, ocular, aural, buccal spray, or nasal spray administration.

In one embodiment, when the pharmaceutical composition is in the form of a tablet, the composition is sufficiently compressible for tablet formation. In one embodiment, the composition can sustain a compression force of at least 10 psi for at least 10 seconds, such as a compression force of at least 100 psi for at least 10 seconds, such as a compression force of at least 1000 psi for at least 10 seconds.

The formulations disclosed herein can be made using at least one method chosen from spray drying, spray granulation, wet granulation; fluid bed granulation, high shear granulation, fluid bed drying, lyophilization, tableting, jet milling, pin milling, wet milling, rotogranulalion, freezer milling, and spray coating.

In one embodiment, the composition comprises a polymeric blend. In one embodiment, the at least one carboxy alkylcellulose ester is a polymer representing one or more components of the blend. In one embodiment, the carboxy alkylcellulose ester polymers are anionic (C₂-C₄) cellulose esters having an acid number ranging from 30 to 120. In another embodiment, the carboxy alkylcellulose esters are anionic C₂ cellulose esters having an acid number ranging from 40 to 100. The blend can optionally include other components, such as one or more of any water soluble, pH sensitive, or water insoluble polymer useful in enteric coatings. Examples of useful water soluble polymers include, but are not limited to, hydroxypropyl cellulose, hydroxypropyl methyl cellulose, or methyl cellulose. Examples of pH sensitive polymers include, but are not limited to, cellulose acetate phthalate, cellulose acetate succinate, cellulose acetate trimellitate, or hydroxypropyl methyl cellulose phthalate. Examples of useful water insoluble polymers include, but are not limited to, cellulose acetate, cellulose acetate propionate, or cellulose acetate butyrate. Those skilled in the art will recognize that the ratio of the blend components is dependent upon the individual formulation and the desired release rate of the therapeutic agent.

In one embodiment, the blend comprises a film, which can be prepared by any method known in the art, such as solvent casting, co-precipitation, freeze drying, and spray drying.

In one embodiment, the carboxy alkylcellulose ester aids in the release of therapeutic agents from a solid core. Such carboxy alkylcellulose esters can be anionic, cationic, or zwitterionic C₂-C₈ cellulose esters, such as anionic C₂-C₄ cellulose esters having an acid number from about 40 to about 120.

The carboxy alkylcellulose esters can be incorporated into the solid core along with the therapeutic agent by a number of techniques well known to those skilled in the art. In one embodiment, the solid core comprises one or more oxidized cellulose ester, a pharmaceutically acceptable carrier, and a therapeutically effective amount of therapeutic agent. A film coating optionally surrounds the solid core. These solid cores can be in the form of, by way of example and without limitation, chewable bar, capsule, fiber, film, gel, granule, chewing gum, pellet, powder, tablet, stick, strip and wafer.

In one embodiment, the composition comprises a solid dispersion (also known as solid solution), i.e., the at least one pharmaceutically active agent is dispersed in a solid dispersant. Without wishing to be bound by any theory, the solid dispersant may disrupt the crystal structure of the drug, thereby reducing the crystal lattice energy. The energy required to dissolve the drug substance can be reduced, which may result in increased dissolution rates, and thus, the bioavailability of the agent.

In one embodiment, the solid dispersant comprises the at least one carboxyalkylcellulose ester. In on embodiment, the carboxyalkylcellulose ester dispersant can be blended with other conventional solid dispersants, such as hydrophilic compounds or polymers. Exemplary dispersants include physiologically inert compounds that are water soluble, e.g., polyethylene glycols, such as those disclosed in U.S. Pat. No. 6,197,787. Other solid dispersants that may be combined with the at least one carboxyalkylcellulose ester include cellulose and its derivatives, such as microcrystalline cellulose (MCC), carboxymethylcellulose (CMC), hydroxypropyl methylcellulose (HPMC), hydroxypropyl methylcellulose acetate succinate (HPMCAS), hydroxypropyl methylcellulose phthalate (HPMCP), cellulose acetate phthalate (C-A-P), cellulose acetate (CA), ethyl cellulose, and methyl cellulose. Anionic cellulose derivatives may also be used (e.g. CMC, HPMCAS, HPMC).

Another embodiment disclosed herein provides a method of treating a mammal in need thereof with a sustained release pharmaceutical composition, comprising:
administering to the mammal in need of treatment the sustained release pharmaceutical composition comprising:
   a therapeutically effective amount of at least one pharmaceutically active agent, and
   at least one carboxyalkylcellulose ester comprising an anhydroglucose repeat unit having the structure: 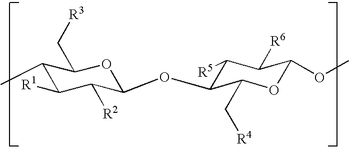
wherein:
R¹-R⁶ are each independently selected from —OH, —OC(O)(alkyl), and —O(CH₂)ₓC(O)OH, and pharmaceutically acceptable salts thereof, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75, and
   allowing sustained release of the at least one pharmaceutically active agent.

In one embodiment, the sustained release follows zero order kinetics.

In one embodiment, the terms “treatment” and its cognates (e.g., “therapeutic method”) refer to both therapeutic treatment and prophylactic/preventative measures. Those in need of treatment may include humans or animals already having a particular medical disease as well as those at risk for the disease (i.e., those who are likely to ultimately acquire the disorder). A therapeutic method results in the prevention or amelioration of symptoms or an otherwise desired biological outcome and may be evaluated by improved clinical signs, delayed onset of disease, reduced/elevated levels of lymphocytes and/or antibodies, etc.

Actual dosage levels of active ingredients in the pharmaceutical compositions described herein may be varied so as to obtain an amount of the active compound(s) that is effective to achieve the desired therapeutic response for a particular patient, compositions, and mode of administration. The terms “therapeutically effective dose” and “therapeutically effective amount” refer to that amount of a compound that results in prevention or amelioration of symptoms in a patient or a desired biological outcome, e.g., improved clinical signs, delayed onset of disease, reduced/elevated levels of lymphocytes and/or antibodies, etc. The effective amount can be determined as described herein. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. In one embodiment,.the data obtained from the assays can be used in formulating a range of dosage for use in humans.

Generally dosage levels of about 0.1 μg/kg to about 50 mg/kg, such as a level ranging from about 5 to about 20 mg of active compound per kilogram of body weight per day, can be administered topically, orally or intravenously to a mammalian patient. Other dosage levels range from about 1 μg/kg to about 20 mg/kg, from about 1 μg/kg to about 10 mg/kg, from about 1 μg/kg to about 1 mg/kg, from 10 μg/kg to 1 mg/kg, from 10 μg/kg to 100 μg/kg, from 100 μg to 1 mg/kg, and from about 500 μg/kg to about 5 mg/kg per day. If desired, the effective daily dose may be divided into multiple doses for purposes of administration, e.g., two to four separate doses per day. In one embodiment, the pharmaceutical composition can be administered once per day.

### EXAMPLES

Degree of substitution (DS) was determined by ¹H NMR or GC. For the carboxy(C₁-C₃)alkylcellulose esters exemplified herein, a GC method was used to determine acetyl, propionyl, and butyryl. The DS values were calculated by converting the acid number to percent carboxymethyl and using this along with the GC weight percents of acetyl, propionyl, and butyryl.

Specifically, the acetyl, propionyl, and butyryl weight percents were determined by a hydrolysis GC method. In this method, about 1 g of ester was weighed into a weighing bottle and dried in a vacuum oven at 105° C. for at least 30 minutes. Then 0.500±0.001 g of sample was weighed into a 250 mL Erlenmeyer flask. To this flask was added 50 mL of a solution of 9.16 g isovaleric acid, 99%, in 2000 mL pyridine. This mixture was heated to reflux for about 10 minutes, after which 30 mL of isopropanolic potassium hydroxide solution was added. This mixture was heated at reflux for about 10 minutes. The mixture was allowed to cool with stirring for 20 minutes, and then 3 mL of concentrated hydrochloric acid was added. The mixture was stirred for 5 minutes, and then allowed to settle for 5 minutes. About 3 mL of solution was transferred to a centrifuge tube and centrifuged for about 5 minutes. The liquid was analyzed by GC (split injection and flame ionization detector) with a 25M×0.53 mm fused silica column with 1 μm FFAP phase.

The weight percent acyl was calculated as follows, where:
Cᵢ=concentration of I (acyl group)
Fᵢ=relative response factor for component I
Fₛ=relative response factor for isovaleric acid
Aᵢ=area of component I
Aₛ=area of isovaleric acid
R=(grams of isovaleric acid)/(g sample)
Cᵢ=((Fᵢ*Aᵢ)/Fₛ*Aₛ))*R*100

The acid number of the carboxy(C₁-C₃)alkylcellulose esters was determined by titration as follows. An accurately weighed aliquot (0.5-1.0 g) of the carboxy(C₁-C₃)alkylcellulose ester was mixed with 50 mL of pyridine and stirred. To this mixture was added 40 mL of acetone followed by stirring. Finally, 20 mL of water was added and the mixture stirred again. This mixture was titrated with 0.1 N sodium hydroxide in water using a glass/combination electrode. A blank containing 50 mL of pyridine, 40 mL of acetone, and 20 mL of water was also titrated. The acid number was calculated as follows where:
Ep=mL NaOH solution to reach end point of sample
B=mL NaOH solution to reach end point of blank
N=normality of sodium hydroxide solution
Wt.=weight of carboxyalkyl cellulose ester titrated.
Acid Number (mg KOH/g sample)=((Ep-B)*N*56.1)/Wt.

### Example 1

This Example describes comparative testing between compositions containing aspirin and a prior art cellulose versus aspirin-containing compositions comprising carboxyalkyl cellulose esters disclosed herein.

Table 1 lists the compositions of the Comparative Formulations C1-C8.

TABLE 1

| | | | Aspirin | | Mg |
|---|---|---|---|---|---|
| Comparative | | | unless | | stearate/carbon |
| Formulations: | Binder | Binder, g | noted, g | | black blend, g |
| C1 | Aspirin Tablet, with microcrystalline | 0 | 180-200 | mg | 0.00 |
| | cellulose (“MCC”) (Sigma Aldrich | | | | |
| | Chemical Co.) | | | | |
| C2 | Aspirin Tablet with Cellulose acetate | 6.00 | 1.5 | | 0.04 |
| | (“CA-398-30,” Eastman Chemical | | | | |
| | Company) | | | | |
| C3 | Aspirin Tablet with Sodium- | 2.80 | 0.91 | | 0.03 |
| | Carboxymethyl cellulose, Acid | | | | |
| | Number = 65 (Cekol, (“Na-CMC”), | | | | |
| | Noviant) | | | | |
| C4 | Aspirin Tablet with Cellulose acetate | 6.00 | 1.5 | | 0.04 |
| | phthalate (“C-A-P,” Eastman | | | | |
| | Chemical Company) | | | | |
| C5 | Aspirin Tablet with Directly Oxidized | 3.70 | 1.50 | | 0.03 |
| | Cellulose Acetate, Acid Number = 88 | | | | |
| | (“Ox-CA,” U.S. App. Pub. No. | | | | |
| | 2005/0192434 | | | | |
| C6 | Aspirin Tablet with Directly Oxidized | 2.99 | 1.00 | | 0.03 |
| | Cellulose Acetate Propionate, Acid | | | | |
| | Number = 67 (“Ox-CAP,” U.S. App. | | | | |
| | Pub. No. 2005/0192434 | | | | |
| C7 | Aspirin Tablet with Directly Oxidized | 2.98 | 0.94 | | 0.03 |
| | Cellulose Acetate Butyrate, Acid | | | | |
| | Number = 32 (“Ox-CAB,” U.S. App. | | | | |
| | Pub. No. 2005/0192434 | | | | |

| | | | | |
|---|---|---|---|---|
| C8 | Ibuprofen Tablet with | 4.68 | Ibuprofen | 0.03 |
| | Hydroxypropylmethylcellulose | | 1.18 | |
| | (“HPMC,” Sigma Aldrich Chemical | | | |
| | Co.) | | | |

The directly oxidized cellulose ester of Formulations C5-C7 were prepared in accordance with the methods described in U.S. Application Publication No. 2005/0192434, the disclosure of which is incorporated herein by reference, which describes a method for converting a primary alcohol to a formyl or carboxylate substituent, or mixture thereof, comprising adding an amino substituted cyclic nitroxyl derivative, a primary oxidant, and a terminal oxidant to a cellulose mixture having a pH of less than 4 to form a reaction mixture, wherein the cellulose mixture comprises a C₂-C₁₂ alkyl acid, water, and a cellulose interpolymer comprising anhydroglucose units having C₆ hydroxyl groups; and passing of a reaction period sufficient to effect conversion of a C₆ hydroxyl to a formyl group or a carboxy group and thus produce an oxidized cellulose interpolymer. More specifically, the ester of Formulations C5-C7 were prepared by adding a 4-substituted piperidine nitroxyl derivative wherein the substituent is capable of hydrogen bonding, a primary oxidant, and a terminal oxidant to a mixture to form a reaction mixture, wherein the mixture has a pH of less than about 4 and includes a compound containing a primary alcohol functional group; passing of a reaction period sufficient to effect conversion of the primary alcohol functional group. The samples were converted to the oxidized cellulose ester using a high hydroxyl cellulose acetate (e.g., a degree of substitution of hydroxyl of 0.1 or more), cellulose acetate propionate or cellulose acetate butyrate, in which the hydroxyl groups were largely converted to carboxyl groups.

Table 2 lists compositions prepared according to the present disclosure, i.e., Formulations E1-E 16. The compounds CMCA, CMCAP, and CMCAB were prepared in accordance with the methods described in U.S. Pat. Nos. 5,668,273 and 5,994,530, the disclosures of which are incorporated herein by reference.

TABLE 2

| | | | | | Mg |
|---|---|---|---|---|---|
| | | | | | stearate/ |
| | | | | Vitamin | carbon |
| Example | Binder | Binder, g | Aspirin, g | E TPGS, g | black blend, g |
| E1 | Carboxymethyl cellulose acetate | 4.51 | 1.50 | 0.00 | 0.03 |
| | (“CMCA”), Acid number = 67 | | | | |
| | (degrees of substitution(DS), | | | | |
| | CM = 0.32, DS Ac = 2.58, DSOH = 0.1) | | | | |
| E2 | Carboxymethyl cellulose acetate | 3.01 | 0.99 | 0.00 | 0.03 |
| | propionate (“CMCAP”), Acid Number = 67, | | | | |
| | (DS, Pr = 2.4, CM = 0.32, | | | | |
| | Ac = 0.18, OH = 0.1) | | | | |
| E3 | Carboxymethyl acetate | 4.65 | 1.51 | 0.00 | 0.03 |
| | butyrate(“CMCAB”) (DS, Bu = 1.85, | | | | |
| | Ac = .33, OH = .3, CM = 0.32 (Acid | | | | |
| | Number = 60) “0” | | | | |
| E4 | CMCAB “1” | 4.20 | 1.5 | 0.30 | 0.03 |
| E5 | CMCAB “2, 4, 10, 15” | 4.66 | 1.05 | 0.30 | 0.03 |
| E6 | CMCAB “3” | 4.43 | 1.13 | 0.47 | 0.03 |
| E7 | CMCAB “5” | 4.23 | 1.85 | 0.00 | 0.03 |
| E8 | CMCAB “6” | 4.80 | 1.20 | 0.00 | 0.03 |
| E9 | CMCAB “7” | 5.10 | 0.63 | 0.33 | 0.03 |
| E10 | CMCAB “8” | 5.41 | 0.69 | 0.00 | 0.03 |
| E11 | CMCAB “9” | 4.45 | 1.44 | 0.16 | 0.03 |
| E12 | CMCAB “11” | 4.20 | 1.19 | 0.63 | 0.03 |
| E13 | CMCAB “12” | 4.75 | 0.82 | 0.45 | 0.03 |
| E14 | CMCAB “13” | 5.00 | 0.82 | 0.15 | 0.03 |
| E15 | CMCAB “14” | 4.53 | 0.94 | 0.64 | 0.03 |
| E16 | CMCAB “16” | 4.50 | 1.50 | 0.00 | 0.03 |

Unless otherwise stated, physical blends were prepared by grinding the cellulosic binder with the aspirin and vitamin E-TPGS in a SPEX™ liquid nitrogen Freezer Mill for 6 minutes at 75% maximum speed. Pigmented magnesium stearate (0.04 g), (0.13 g carbon black to 1.0 g Mg stearate) was post-added to the finely ground powder and mixed until an even pale gray color was achieved, insuring even mixing of the lubricant. The C2, C4 and C5 tablets were compressed with a seven tablet press set at 5000 psi (pounds per square inch) for 15 seconds. The E1-E16, C1, C3, C6-C7 tablets were compression molded using a commercial TEVO™ single pill press at a compression force of 2000 pounds for 10 seconds. The tablets were capable of being pressed up to 4500 pounds for 10 seconds in the TEVO™ single pill press without significant changes in the dissolution profiles. All tablets except C3, C4, E4, and E11 (Aspirin with Na-CMC, C-A-P, CMCAB with 5% Vitamin E TGPS, and CMCAB with 5% Vitamin E TGPS, respectively) had low friability. Formulations C3, C4, E4, and E11 were not suitable formulations for compression tablets and were quite friable, regardless of the pressure applied to the pill presses. These friable formulations can be useful for rapid disintegration yet allow sustained delivery.

The dissolution tests were done using a USP #2 calibrated apparatus Varian VK 7000 with Teflon paddles. The pills were added to 900 ml of USP 1.2 pH buffer or to 900 ml of USP pH 6.8 buffer. The buffer solutions had each been degassed at 41° C. through a 0.45 micron hydrophilic polypropylene filter and held under vacuum for an additional 5 minutes. After the solutions were added to the dissolution vessels, the solutions were held at 37.3° C. in the water bath for 30 minutes to achieve constant temperature, prior to the addition of the tablets. The tablets were weighted down with a Varian 3-pronged capsule weight. At the beginning of each run, the tablets were allowed to sink to the bottom of the 1000 ml vessel, the stirrers were turned on at 50 rpm and samples taken as a function of time, using polypropylene syringes. The samples were filtered through 0.45 micron filters and immediately analyzed for the amount of aspirin in the solution. The wavelength for measuring the amount of aspirin (salicylic acid acetate) at pH 1.2 was 279 nm, which was the wavelength for equivalent molar absorptivities for both salicylic acid and aspirin using a Varian UV-VIS Spectrophotometer and quartz absorption cells. Similarly, the wavelength used to measure the concentration of the aspirin in pH 6.8 buffer was 267 nm, which was the wavelength where the molar absorptivity for salicylic acid and aspirin are equivalent at pH 6.8. This allowed accurate measurement of the release rate profile without having to worry about the degradation of the aspirin to salicylic acid with time. Each set of experiments had appropriate standards for reference for quantitative analysis. In the cases where the samples contained Vitamin E-TPGS, HPLC with UV detection was done to analyze for aspirin and salicylic acid.

The C1 tablet and the C3 tablets completely disintegrated after a few minutes at both pH 1.2 and pH 6.8. In contrast, the C2 tablets with aspirin did not appear to undergo physical changes throughout the course of the experiment. The C4 tablets completely disintegrated after 1 hour in both the pH 1.2 and the pH 6.8 buffer solutions. However, the buffer solution for C4 at pH 1.2 remained cloudy, while the pH 6.8 buffer solution became clear after 1 hour. During the first hour of dissolution, it did not appear that the C5 tablet (prepared with Ox-CA) was responding to the higher pH environment, but only to the lower pH solution. At pH 1.2, the C5 (Ox-CA) tablets formed cracks in solution. At pH 6.8, the tablets retained their original shape during the first hour. However, after 3 hours, the C5 tablets in pH 6.8 buffer solution had almost completely dissolved leaving a clear solution. There were no change in the C6 tablets after 6 hours at pH 1.2, but at pH 6.8 the tablets had completely disintegrated. The C7 tablets disintegrated at pH 6.8 but did not dissolve. There were no apparent changes at pH 1.2. The experiments were carried out for 6-22 hours.

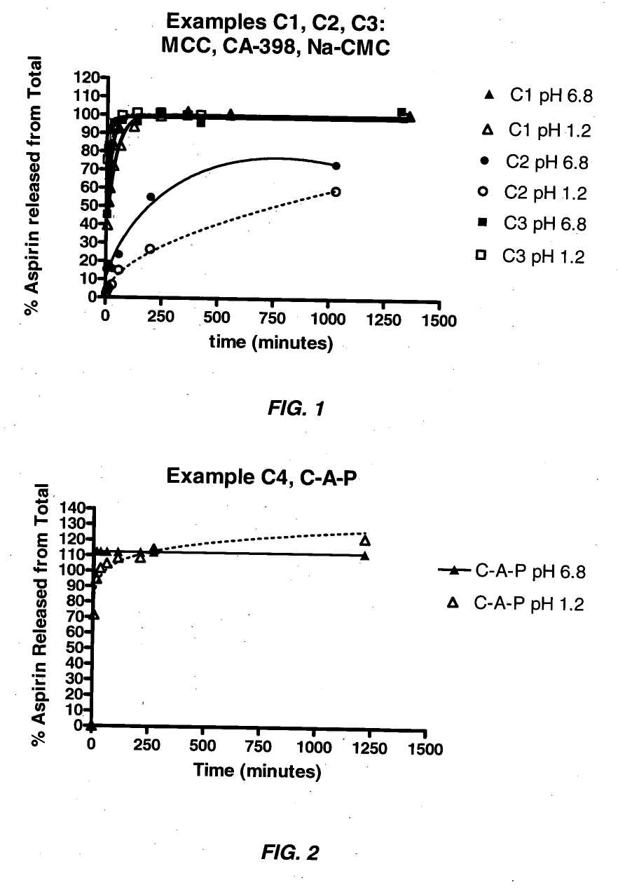

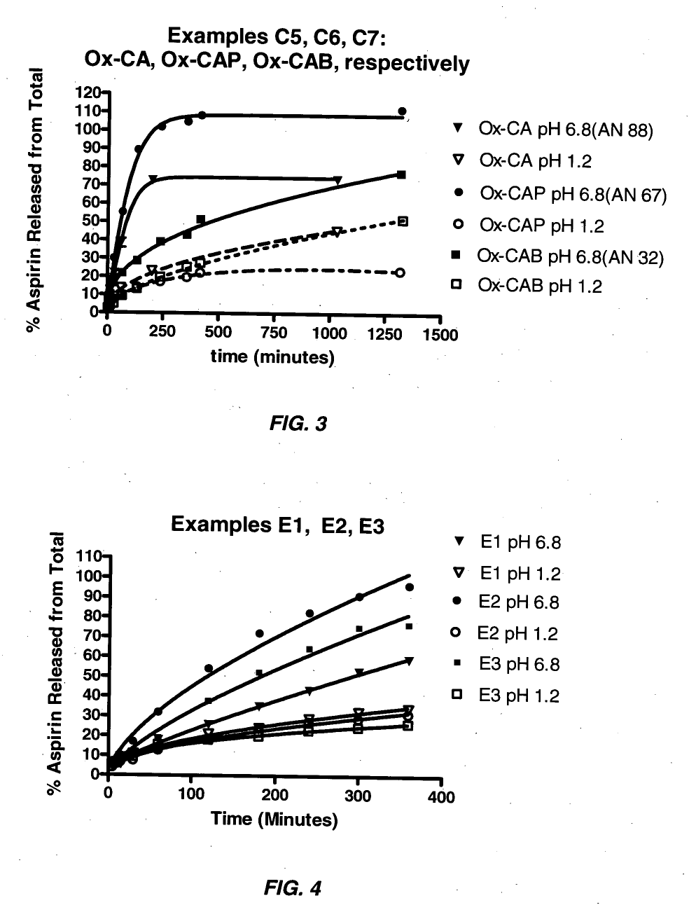

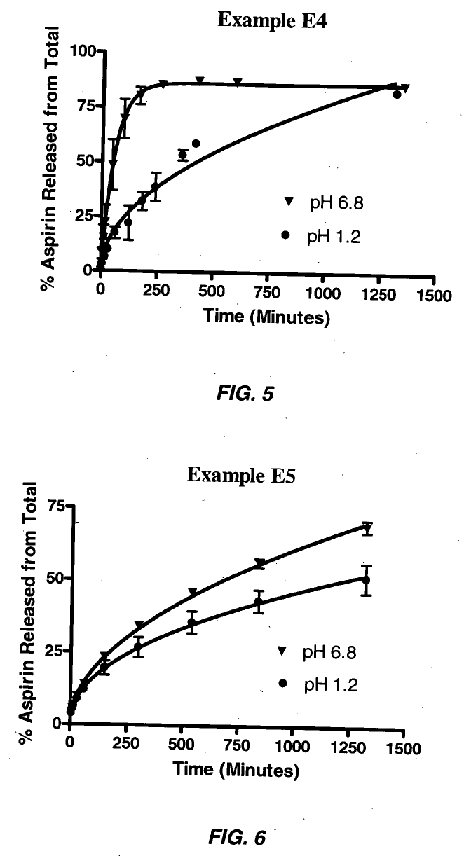

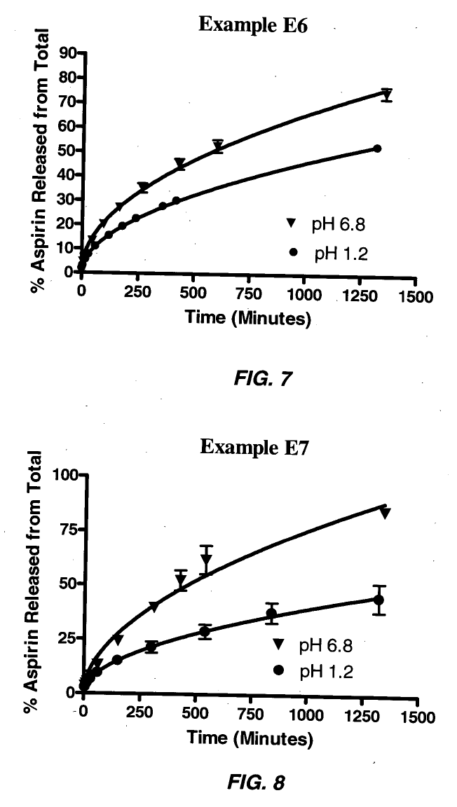

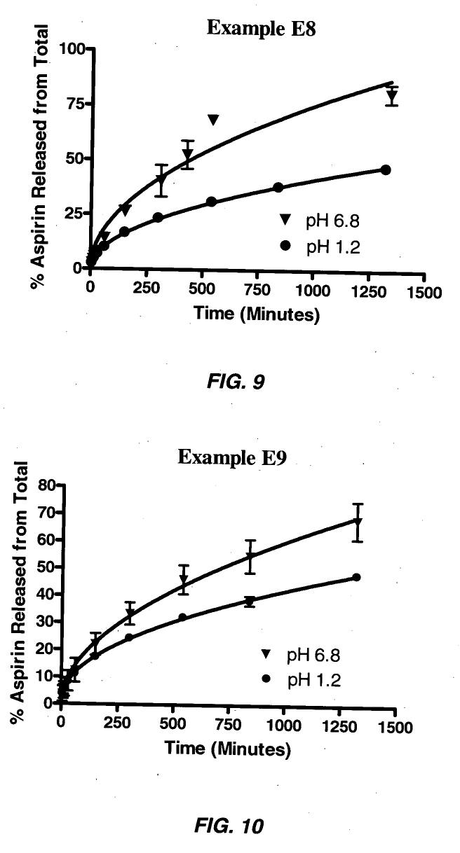

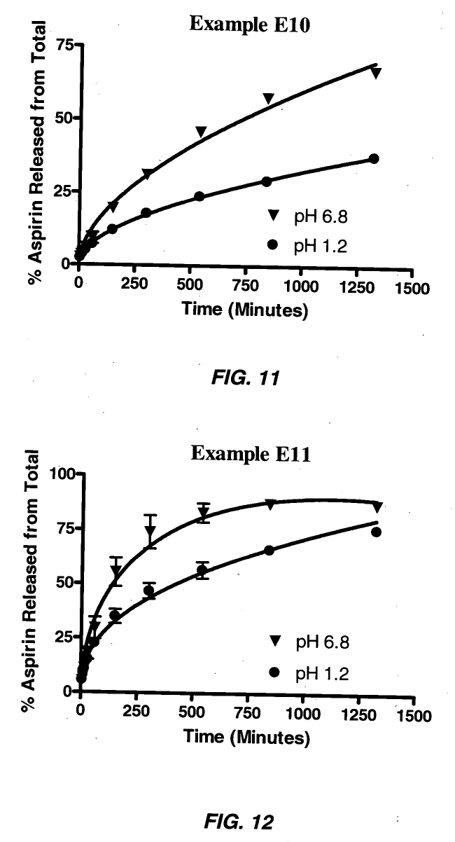

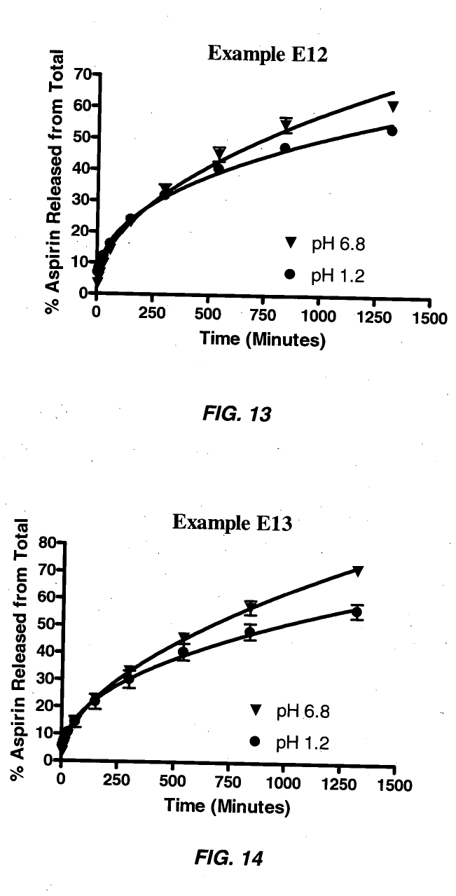

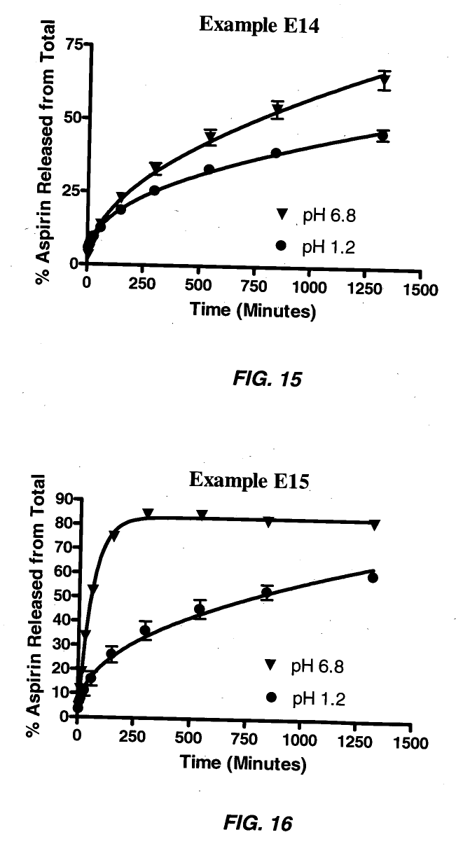

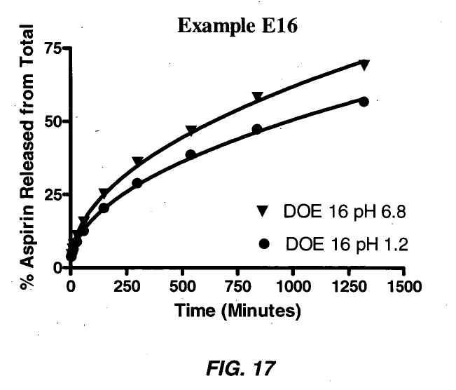

FIGS. 1-3 are plots of aspirin released (y-axis) versus time (minutes, x-axis) for Comparative Formulations C1-C3, Formulation C4, and Formulations C5-C7, respectively. FIGS. 4-17 are plots of aspirin released (y-axis) versus time (minutes, x-axis) for Formulations E1-E3, and E4-E16, respectively.

In the above comparative examples, it was noted surprisingly that neither C-A-P in Formulation C4, nor Na-CMC in Formulation C3 provided sustained release even at low pH. C-A-P in Formulation C4 did not form a good direct compression binder with aspirin or other actives. Cellulose acetate in Formulation C2 slowed the release of aspirin down in both pH environments according to the relative solubility of aspirin. The higher pH formulations had higher concentrations and the lower pH formulations had lower overall concentrations. However, the total release of aspirin from 0-3 hours at pH 1.2 was higher in the CA-398 tablets than in CMCAB aspirin tablets (e.g., Formulations E2 or E3, for the comparable tablet) and the total release was also much lower over 24 hours at pH 6.8 for the CA-398 tablet than for CMCAB aspirin in the same time. Neither Ox-CA in Formulation C5 nor Ox-CAP in Formulation C6 gave sustained release for more than 3 hours at pH 6.8. The differentiation in release of aspirin at pH 1.2 and pH 6.8 was not as pronounced as it was for formulations with either CMCA, CMCAP or CMCAB, as shown by Formulations E1, E2, and E3.

In comparison, the compositions prepared according to the present disclosure demonstrated substantially linear slow release by the carboxyalkyl cellulose esters. Depending of the type of substituents on the carboxyalkyl cellulose esters, the release rate of aspirin at elevated pH could be moderate (6 hours) or slow (over 24 hours). Formulations E4-E16 also demonstrated the by varying the use of Vitamin E TGPS in the formulation, one could vary the pH sensitivity of the release of aspirin as well as the rate of release as a function of pH.

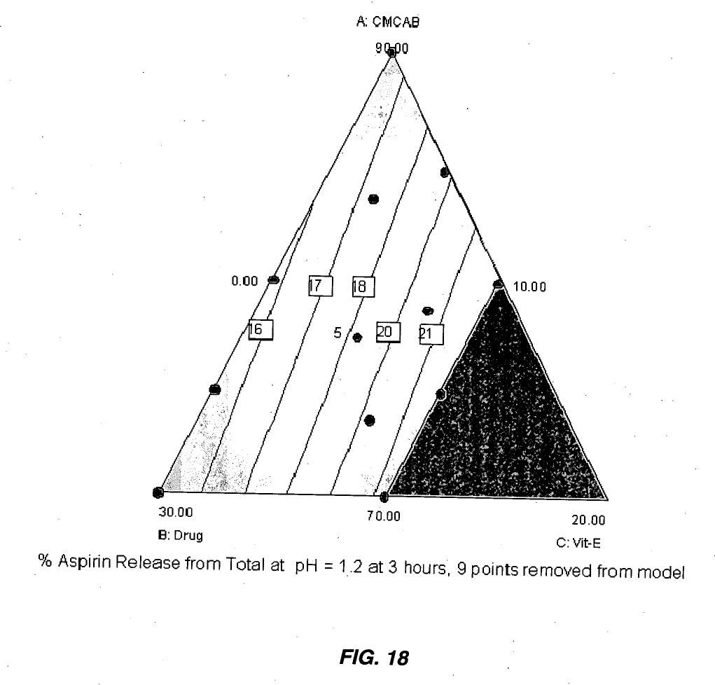

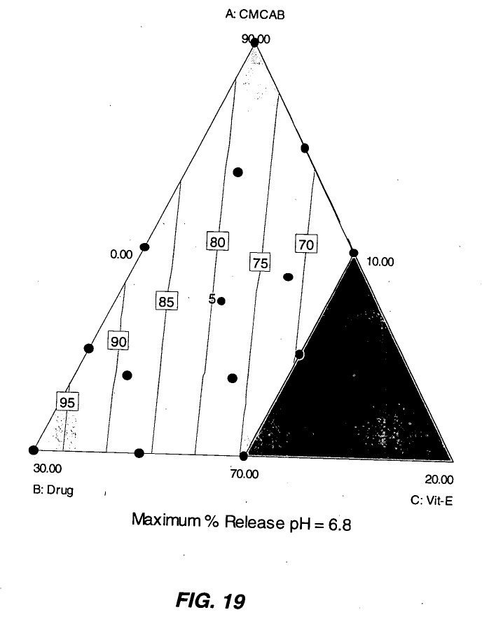

FIG. 18 is a ternary plot showing statistical results for the release of aspirin at pH 1.2 for up to 3 hours. FIG. 19 is a ternary plot showing statistical results for the release of aspirin at pH 6.8 after 24 h. In these plots, the far left bottom corner shows that the minimum release rate occurs with no Vit-E TGPS present. In addition a desirability function was run on the model developed to find the most preferred embodiments of the invention. The desirability was set at no more than 20% of aspirin released at pH 1.2 for 3 hours and greater than or equal to 70% aspirin release in 24 hours.

### Example 2

Exemplary formulations with aspirin are shown in the Table 3 below. In these formulations, magnesium stearate was added as a mold release agent.

TABLE 3

| | | | Predicted % | Predicted |
|---|---|---|---|---|
| | | | release at pH | release at pH |
| % | % Vit | % | 6.8 over 24 | 1.2 over 3 |
| Drug | E-TPGS | CMCAB | hours | hours |
| 30.00 | 0.00 | 70.00 | 98 | 14 |
| 17.37 | 0.00 | 82.63 | 86 | 16 |
| 12.67 | 0.00 | 87.33 | 81 | 16 |

The amount of release in the tablet was targeted at levels of at least 70% of the active released in 24 hours. Therefore, all the tablets tested using the carboxyalkyl cellulose esters as the tablet binder, fell into that category. However, not all slowed down or prevented substantial amounts of aspirin from dissolving at pH 1.2 over a 3 hour time period. From the table, exemplary aspirin formulations ranged from 12.5%-30% aspirin and 87.5%-70% CMCAB.

### Example 3

In this Example, the sustained delivery of other pharmaceutically active agents was tested. Table 4 below lists Formulations E17-E23 containing other pharmaceutically active agents.

TABLE 4

| | | | | Mg |
|---|---|---|---|---|
| | Binder, g | | | stearate/carbon |
| Example | CMCAB | Active | Active, g | black blend, g |
| E17 | 2.96 | Trimethoprim | 0.89 | 0.03 |
| E18 | 2.99 | Sulfamethizole | 1.01 | 0.03 |
| E19 | 3.00 | Levofloxacin | 1.12 | 0.03 |
| E20 | 2.98 | Ibuprofen | 1.02 | 0.03 |
| E21 | 2.99 | Amiloride HCl | 1.02 | 0.03 |
| E22 | 2.98 | Fluconazole | 1.03 | 0.03 |
| E23 | 2.94 | Fexafenadine HCl | 1.02 | 0.03 |

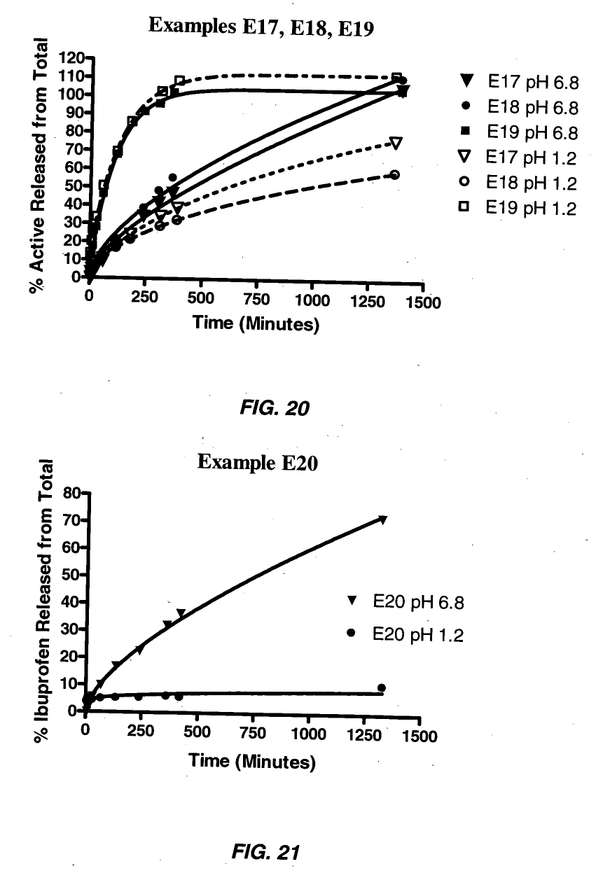

FIG. 20 is a plot of trimethoprim, sulfamethizole, and levofloxacin released (y-axis) versus time (minutes, x-axis) for Formulations E17-19. As can be seen from FIG. 20, while levofloxacin has a very rapid release at both pH 1.2 and 6.8, the rate of release could be slowed down by using more CMCAB in the formulation. The levofloxacin was so hydrophilic that it acted as a tablet dispersant and caused the tablet to disintegrate very quickly. However, even with levofloxacin, the rate was sustained over 4 hours, rather than immediate release. Using triethyl citrate or other water-insoluble plasticizer would slow the release rate down, if desired.

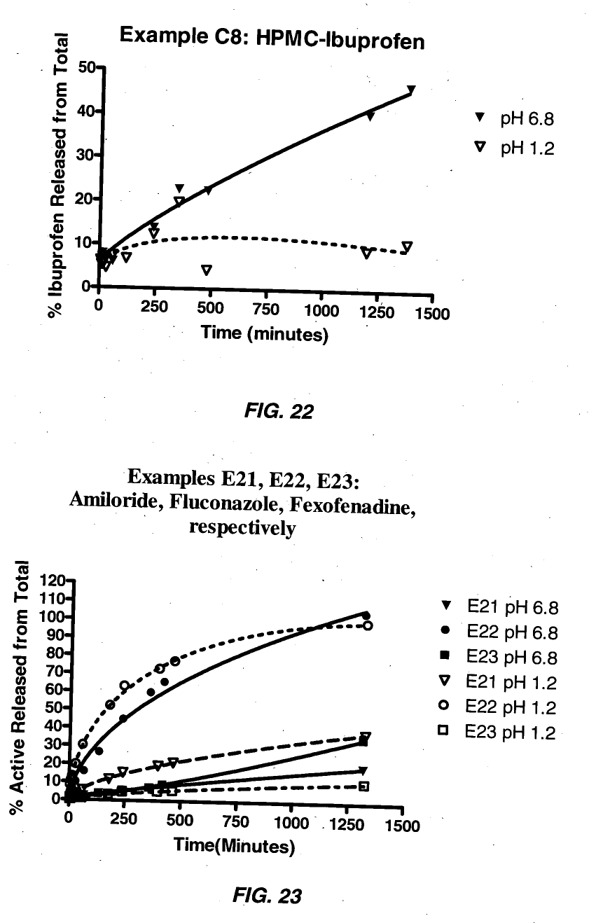

FIG. 21 is a plot of ibuprofen released (y-axis) versus time (minutes, x-axis) for Formulation E20. FIG. 21 is a plot of ibuprofen released (y-axis) versus time (minutes, x-axis) for Comparative Formulation C8 in Example 1. FIGS. 21 and 22 show similar behaviors at pH 1.2 (very little release) and very slow sustained release in both cases at pH 6.8. However, it must be noted that in both cases, ibuprofen is highly insoluble at pH 1.2 and very soluble at pH 6.8. Therefore, the fact that little dissolved in either case, was not surprising. However, it was surprising that HPMC, a very hydrophilic compound, retarded the dissolution of ibuprofen at pH 6.8 too much when used as the compression media. Thus, HPMC was unable to provide sustained release needs in excess of 50% of the total active drug in 24 hours.

FIG. 23 is a plot of amiloride, fluconazole, and fexofenadine released (y-axis) versus time (minutes, x-axis) for Formulations E21-23, respectively. FIG. 23 shows that similarly, fluconazole showed little pH dependence, being highly water soluble. CMCAB was able to provide near zero order release for up to 500 minutes at 25% active, but more CMCAB in the formulation would be preferable. The sustained release of amiloride could be improved by the use of a surfactant or Vit-E TPGS to solubilize the material.

Ibuprofen, sulfamethizole, and trimethoprim gave extended release formulations that were close to zero order for up to 22 hours at pH 6.8 while having low total release at pH 1.2

### Example 4

This Example describes the preparation of an extended release formulation made from a water soluble active and CMC esters incorporated in a polymer blend.

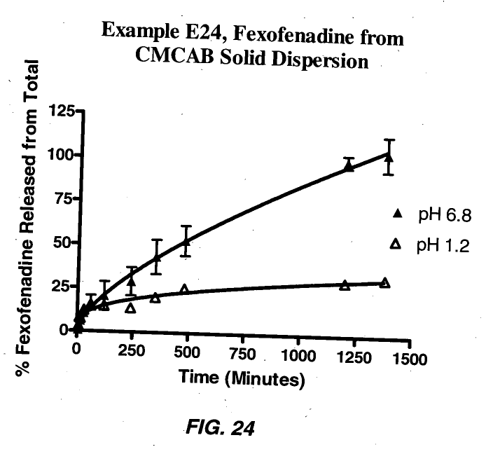

6.0069 g CMCAB was mixed with 1.4937 g Fexofenadine HCl and 10.7693 g ethanol. The clear solution was allowed to dry into a clear film. The amorphous compatible film was then ground in a SPEX™ liquid nitrogen Freezer Mill for 6 minutes at 75% maximum speed. Torpac Inc. #2 Gelatin capsules were filled with the powdered polymer blend and tested using a Varian VK7000 USP II dissolution device at pH 1.2 and pH 6.8. The dissolution results are shown in FIG. 24, which is a plot of fexofenadine released from CMCAB solid dispersion (y-axis) versus time (minutes, x-axis) for Formulation E24 in Example 4. FIG. 24 shows nearly zero order release over 24 hours with 100% of the active released. By varying the way the carboxyalkyl cellulose ester is mixed with the active, the rate of release can be varied so that one can achieve near zero order release of the active over a long period of time.

This Example demonstrates that such polymer blends can be made by solvent casting, co-precipitation, freeze drying, spray drying or other methods known in the art to maintain the integrity of the blend by not allowing the individual components separate.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A sustained release pharmaceutical composition comprising:
at least one pharmaceutically active agent, and
at least one carboxyalkylcellulose ester comprising an anhydroglucose repeat unit having the structure: 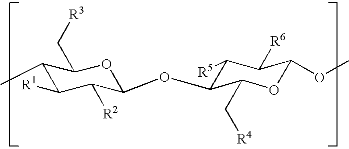
wherein:
R¹-R⁶ are each independently selected from —OH, —OC(O)(alkyl), and —O(CH₂)ₓC(O)OH, and pharmaceutically acceptable salts thereof, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75, and
wherein in pharmaceutically acceptable media, the composition exhibits sustained release of the at least one pharmaceutically active agent.

2. The composition according to claim 1, wherein the sustained release comprises substantially continuous release of the at least one pharmaceutically active agent over time.

3. The composition according to claim 1, wherein the sustained release of the at least one pharmaceutically active agent occurs over a time of at least 4 hours.

4. The composition according to claim 1, wherein the sustained release of the at least one pharmaceutically active agent occurs over a time ranging from 12-24 h.

5. The composition according to claim 1, wherein the sustained release of the at least one pharmaceutically active agent occurs over a time ranging from 6-12 h.

6. The composition according to claim 1, wherein the sustained release of the at least one pharmaceutically active agent occurs over a time of at least 24 h.

7. The composition according to claim 1, wherein the sustained release follows zero order kinetics such that the composition exhibits zero-order release.

8. The composition according to claim 7, wherein the zero-order release is indicated by a substantially linear plot of released pharmaceutically active agent over time, where “substantially linear” refers to a correlation coefficient (R) of at least 0.8.

9. The composition according to claim 1, wherein the at least one pharmaceutically active agent is soluble in pharmaceutically acceptable media, as determined by the Biopharmaceutics Classification System.

10. The composition according to claim 1, wherein the at least one pharmaceutically active agent is chosen from aspirin, ibuprofen, fexofenadine, trimethoprim, sulfamethizole, amiloride, fluconazole, and fexofenadine, and salts thereof.

11. The composition according to claim 1, further comprising at least one plasticizer.

12. The composition according to claim 11, wherein the at least one plasticizer is Vitamin E TPGS.

13. The composition according to claim 1, wherein the —OC(O)(alkyl) is chosen from —OC(O)(C₁-C₂₁ alkyl).

14. The composition according to claim 1, wherein the —OC(O)(alkyl) is chosen from —OC(O)(C₁-C₁₁ alkyl).

15. The composition according to claim 1, wherein the —OC(O)(alkyl) is chosen from —OC(O)(C₁-C₅ alkyl).

16. The composition according to claim 1, wherein the —OC(O)(alkyl) is chosen from —OC(O)(C₁-C₃ alkyl).

17. The composition according to claim 1, wherein the at least one carboxyalkyl cellulose ester is chosen from carboxymethyl cellulose esters.

18. The composition according to claim 17, wherein the at least one carboxyalkyl cellulose ester is carboxymethyl propionate having a degree of substitution per anhydroglucose of —C(O)CH₂CH₃ ranging from 1.5 to 2.7.

19. The composition according to claim 17, wherein the at least one carboxyalkyl cellulose ester is carboxymethyl butyrate having a degree of substitution per anhydroglucose of —C(O)CH₂CH₂CH₃ ranging from 1.5 to 2.7.

20. The composition according to claim 17, wherein the at least one carboxyalkyl cellulose ester is carboxymethyl cellulose acetate propionate having a degree of substitution per anhydroglucose of —C(O)CH₃ ranging from 0.1 to 2.65 and a degree of substitution per anhydroglucose of —C(O)CH₂CH₂H₃ ranging from 0.1 to 2.6.

21. The composition according to claim 17, wherein the at least one carboxyalkyl cellulose ester is carboxymethyl cellulose acetate butyrate having a degree of substitution per anhydroglucose of —C(O)CH₃ ranging from 0.1 to 1.65 and a degree of substitution per anhydroglucose of —C(O)CH₂CH₂H₃ ranging from 0.1 to 2.6.

22. The composition according to claim 1, wherein the composition comprises a polymeric blend.

23. The composition according to claim 1, wherein in pharmaceutically acceptable media, the composition exhibits release of the pharmaceutically active agent at a pH of at least 5.

24. The composition according to claim 1, wherein in pharmaceutically acceptable media, the composition exhibits release of the pharmaceutically active agent at a pH of at least 6.

25. The composition according to claim 1, wherein in pharmaceutically acceptable media, the composition exhibits release of the pharmaceutically active agent at a pH of at least 6.5.

26. The composition according to claim 1, wherein the composition is in the form of a tablet.

27. The composition according to claim 1, wherein the composition is in the form of a solid dispersion.

28. A composition comprising:
at least one pharmaceutically active agent, and
at least one carboxyalkylcellulose ester comprising an anhydroglucose repeat unit having the structure: 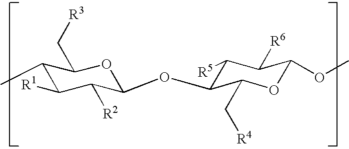
wherein:
R¹-R⁶ are each independently selected from —OH, —OC(O)(alkyl), and —O(CH₂)ₓC(O)OH, and pharmaceutically acceptable salts thereof, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75, and wherein in pharmaceutically acceptable media, the composition exhibits release of the pharmaceutically active agent at a pH of at least 5.

29. A method of treating a mammal in need thereof with a sustained release pharmaceutical composition, comprising:
administering to the mammal in need of treatment the sustained release pharmaceutical composition comprising:
a therapeutically effective amount of at least one pharmaceutically active agent, and
at least one carboxyalkylcellulose ester comprising an anhydroglucose repeat unit having the structure: 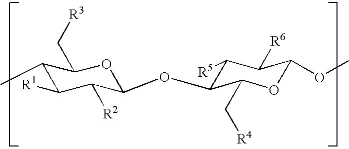
wherein:
R¹-R⁶ are each independently selected from —OH, —OC(O)(alkyl), and —O(CH₂)ₓC(O)OH, and pharmaceutically acceptable salts thereof, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75, and
allowing sustained release of the at least one pharmaceutically active agent.

30. The method according to claim 29, wherein the sustained release follows zero order kinetics.

31. A method of delivering at least one pharmaceutically active agent to a mammal, comprising:
(a) administering to the mammal a therapeutically effective amount of at least one pharmaceutically active agent with at least one carboxyalkylcellulose ester comprising an anhydroglucose repeat unit having the structure: 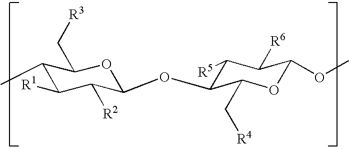
wherein:
R¹-R⁶ are each independently selected from —OH, —OC(O)(alkyl), and —O(CH₂)ₓC(O)OH, and pharmaceutically acceptable salts thereof, wherein x ranges from 1-3,
a degree of substitution per anhydroglucose of —OH ranges from 0.1 to 0.7,
a degree of substitution per anhydroglucose of —OC(O)(alkyl) ranges from 0.1 to 2.7, and
a degree of substitution per anhydroglucose of —O(CH₂)ₓC(O)OH ranges from 0.2 to 0.75;
(b) releasing the at least one pharmaceutically active agent at gastric pH; and
(c) allowing sustained release of the at least one pharmaceutically active agent at intestinal pH

32. The method according to claim 31, wherein the sustained release follows zero order kinetics.
